(19) United States
(12) Patent Application Publication
(10) Pub. No.: US 2010/0029756 A1
(43) Pub. Date: Feb. 4, 2010

(54) NOVEL COMPOSITIONS CONTAINING ISOLATED TETRAMERIC TYPE A PROANTHOCYANADIN AND METHODS OF USE AND MANUFACTURE

(76) Inventors: Michael S. TEMPESTA, El Granada, CA (US); Jan A. Glinski, New Fairfield, CT (US); K. Brian Killday, Ft. Pierce, FL (US)

Correspondence Address: HOGAN & HARTSON LLP, ONE TABOR CENTER, SUITE 1500, 1200 SEVENTEENTH ST, DENVER, CO, 80202, US

(21) Appl. No.: 12/535,438

(22) Filed: Aug. 4, 2009

Related U.S. Application Data
(60) Provisional application No. 61/086,073, filed on Aug. 4, 2008.

(51) Int. Cl. A61K 31/335; C07D 493/18; A61P 9/00

(52) U.S. Cl. 514/452; 549/358

(57) ABSTRACT

The present invention relates to compositions containing an isolated novel tetrameric type A proanthocyanidin isomer having a formula of C₆₀H₄₈O₂₄. The isomer is most preferably isolated from cinnamon (Cinnamomum spp.) and may be used in pharmaceutical compositions as anti-inflammatories, bactericides, antimicrobial formulations, or supplements for general or specific uses.

### RELATED APPLICATION

This application claims priority of U.S. Provisional Application No. 61/086,073 filed Aug. 4, 2008, which is incorporated herein in its entirety by this reference.

### BACKGROUND OF THE INVENTION

1. Field of the Invention

The present invention relates to the extraction, purification and use of isolated proanthocyanadins from cinnamon.

2. State of the Art

Flavonoid compounds are present in all aerial parts of plants, with high concentrations found in the skin, bark, and seeds. Such compounds are also found in numerous beverages of botanical origin, such as tea, cocoa, and wine. The flavonoids are a member of a larger family of compounds called polyphenols. That is, these compounds contain more than one hydroxyl group (OH) on one or more aromatic rings. The physical and chemical properties, analysis, and biological activities of polyphenols and particularly flavonoids have been studied for many years.

Anthocyanins are a particular class of naturally occurring flavonoid compounds that are responsible for the red, purple, and blue colors of many fruits, vegetables, cereal grains, and flowers. For example, the colors of fruits such as blueberries, bilberries, strawberries, raspberries, boysenberries, marionberries, cranberries, elderberries, etc. are due to many different anthocyanins. Over 300 structurally distinct anthocyanins have been identified in nature. Because anthocyanins are naturally occurring, they have attracted much interest for use as colorants for foods and beverages.

Recently, the interest in anthocyanin pigments has intensified because of their possible health benefits as dietary antioxidants. For example, anthocyanin pigments of bilberries (Vaccinium myrtillus) have long been used for improving visual acuity and treating circulatory disorders. There is experimental evidence that certain anthocyanins and other flavonoids have anti-inflammatory properties. In addition, there are reports that orally administered anthocyanins are beneficial for treating diabetes and ulcers and may have antiviral and antimicrobial activities. The chemical basis for these desirable properties of flavonoids is believed to be related to their antioxidant capacity. Thus, the antioxidant characteristics associated with berries and other fruits and vegetables have been attributed to their anthocyanin content.

Proanthocyanidins, also known as “oligomeric proanthocyanidins,” “OPCs,” or “procyanidins,” are another class of naturally occurring flavonoid compounds widely available in fruits, vegetables, nuts, seeds, flowers, and barks. Proanthocyanidins belong to the category known as condensed tannins. They are the most common type of tannins found in fruits and vegetables, and are present in large quantities in the seeds and skins. In nature, mixtures of different proanthocyanidins are commonly found together, ranging from individual units to complex molecules (oligomers or polymers) of many linked units. The general chemical structure of a polymeric proanthocyanidin comprises linear chains of flavonoid 3-ol units linked together through common C(4)-C(6) and/or C(4)-C(8) bonds. ¹³C NMR has been useful in identifying the structures of polymeric proanthocyanidins, and recent work has elucidated the chemistry of di-, tri-, and tetrameric proanthocyanidins. Larger oligomers of the flavonoid 3-ol units are predominant in most plants and are found with average molecular weights above 2,000 Daltons and containing 6 or more monomer units (Newman, et al., Mag. Res. Chem., 25:118 (1987)).

Considerable recent research has explored the therapeutic applications of proanthocyanidins, which are primarily known for their antioxidant activity. These compounds have also been reported to demonstrate antibacterial, anticarcinogenic, antiviral, anti-inflammatory, anti-allergic, and vasodilatory actions. They have also been found to inhibit lipid peroxidation, platelet aggregation, capillary permeability and fragility, and to affect enzyme systems including phospholipase A2, cyclooxygenase and lipoxygenase. For example, proanthocyanidin monomers (i.e., anthocyanins) and dimers have been used in the treatment of diseases associated with increased capillary fragility and have also been shown to have anti-inflammatory effects in animals (Beladi, I. et al., Ann. N.Y. Acad. Sci., 284:358 (1977)). Based on these reported findings, oligomeric proanthocyanidins may be useful components in the treatment of a number of conditions (Fine, A. M., Altern. Med. Rev. 5(2):144-151 (2000)).

Proanthocyanidins may also protect against viruses. In in vitro studies, proanthocyanidins from witch hazel (Hamamelis virginiana) killed the Herpes simplex 1 (HSV-1) virus (Erdelmeier, C. A., et al., Plant Med. June: 62(3):241-5 (1996); DeBruyne, et a.l, J. Nat. Prod. July: 62(7):954-8 (1999)). Another study was carried out to determine the structure-activity relationships of the antiviral activity of various tannins. It was found that the more condensed the chemical structure, the greater the antiviral effect (Takechi, M., et al., Phytochemistry, 24:2245-50 (1985)). In another study, proanthocyanidins were shown to have anti-Herpes simplex activity in which the 50 percent effective doses needed to reduce herpes simplex plaque formation were two to three orders of magnitude less than the 50 percent cytotoxic doses (Fukuchi, K., et al., Antiviral Res., 11:285-298 (1989)).

Cyclooxygenase (COX-1, COX-2) or prostaglandin endoperoxide H synthase (PGHS-1, PGHS-2) enzymes are used to measure the anti-inflammatory effects of plant products (Bayer, T., et al., Phytochemistry, 28:2373-2378 (1989); Goda, Y., et al., Chem. Pharm. Bull., 40:2452-2457 (1992)). COX enzymes are the pharmacological target sites for nonsteroidal anti-inflammatory drugs (Humes, J. L., et al., Proc. Natl. Acad. Sci. U.S.A., 78:2053-2056 (1981); and Rome, L. H., et al., Proc. Natl. Acad. Sci. U.S.A., 72:4863-4865 (1975)). Two isozymes of cyclooxygenase involved in prostaglandin synthesis are cyclooxygenase-1 (COX-1) and cyclooxygenase-2 (COX-2) (Hemler, M., et al., J. Biol. Chem., 25:251, 5575-5579 (1976)). It is hypothesized that selective COX-2 inhibitors are mainly responsible for anti-inflammatory activity (Masferrer, J. L., et al., Proc. Natl. Acad. Sci. U.S.A., 91:3228-3232 (1994)). Flavonoids are being investigated as anti-inflammatory substances, as well as for their structural features for cyclooxygenase (COX) inhibition activity.

Due to the above characteristics and benefits of anthocyanins and proanthocyanidins, much effort has been put forth toward extracting these compounds from fruits, vegetables, and other plant sources. While significant strides have been made in particular in extracting compositions containing numerous anthocyanins and/or proanthocyanidins separated from other naturally occurring materials such as mineral salts, common organic acids such as citric or tartaric acid, carbohydrates, flavonoid glycosides and catechins, numerous individual anthocyanins and/or proanthocyanidins have not been isolated and/or identified due to inherent difficulties.

Indeed, even concentrating and extracting groups of anthocyanins and/or proanthocyanidins can result in a contaminated extractants not preferred for ingestion generally and certainly not of a pharmaceutical grade. For example, one method of extracting anthocyanins employs the addition of bisulfate to form zwitterionic species. The extract is passed through an ion exchange column which adsorbs the zwitterionic anthocyanin adducts, and the adsorbed anthocyanins are eluted from the resin with acetone, alkali, or dimethylformamide (DMF). Disadvantages of this process include the presence of bisulfate, which interferes with adsorption of anthocyanins, thereby requiring multiple column adsorptions. Elution with alkali degrades the anthocyanins considerably, while DMF is not a recognized food additive and therefore must be completely removed before the anthocyanins can be added to any food products.

In order to capture these flavonoid compounds, well-defined and precise processing and separation techniques are used. Even when separated, constituent isomers have historically difficult, if not impossible, to isolate and identify. More recently, however, researchers have successfully isolated a proanthocyanidin trimer from Lindera aggregata, as described in One New A-type Proanthocyanidin Trimer from Lindera aggregata, by C. F. Zhang, et al, Chinese Chemcial Letter, Vol. 14, No. 10, pp. 1033-36, 2003, which is incorporated herein in its entirety by this reference.

Cinnamon (Cinnamomum spp.) has been an item of commerce for human consumption for a very long time, with references in ancient Greek and Latin writings for use as a spice and as a folk medicine for gastrointestinal disorders. Cinnamon has also been the subject of a placebo-controlled clinical study of diabetic patients for 6 weeks to evaluate effects on glucose and lipid metabolism (Khan, A., et al., Diabetes Care, 2003, 26 (12), 3215-3218). Improvements in fasting glucose levels (18-29%), triglycerides (23-30%), LDL cholesterol (7-27%) and total cholesterol (12-26%) were noted over the course of the study in all three dose levels (1, 3 and 6 g/day), suggesting lower doses may also show beneficial effects.

It has been estimated that the average daily human intake of polyphenols from food and spices is 1.5-2.5 grams (Rao, B. S. N., Prabhavati, T., J. Sci. Food Ag., 1982, 33, 89), and there are many common dietary sources for these proanthocyanidin polymers (Hammerstone, J. F., et al., J. Nutr., 2000, 130, 2086S-2092S). Hundreds of polyphenol-based pharmaceutical and dietary supplement products produced from a variety of food and spice sources are available world-wide (bilberry, grape seed, green tea, etc.). Many of these products have been on the market in the U.S., Europe and Asia for decades, and are widely recognized as safe.

Cinnamon contains proanthocyanins and other bioflavonoids that have been shown to inhibit the oxidation of fatty acids by acting as hydrogen atom donors to peroxy radicals (Torel J., et al., Phytochemistry 1986;25:383-385), which can be formed during periods of strenuous exertion. Reduction of free radical damage to lipids, proteins and carbohydrates has also been linked to the lessening of risk of chronic degenerative disease development. Of fifty plant extracts tested, cinnamon was determined to be the most potent in increasing glucose metabolism, as measured by the epididymal fat cell assay (Broadhurst, C. L., et al., In Vitro. J Agric. Food. Chem. 2000; 48: 849-852). Nonetheless, there remains a need for identification of compositions containing one or more phenolic compounds such as proanthocyanidins for use in nutraceuticals and pharmaceuticals which have enhanced activity.

### SUMMARY OF THE INVENTION

The present invention relates to a composition containing an isolated and novel tetrameric, type A proanthocyanidin isomer having a formula of C₆₀H₄₈O₂₄, shown below,

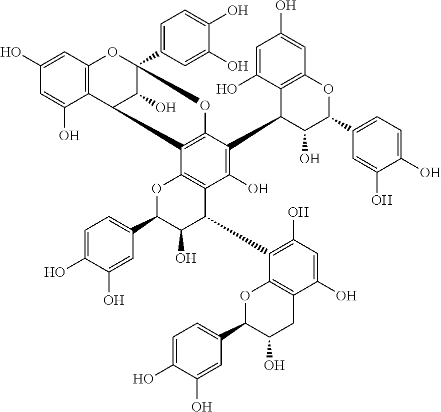

and according to the mass spectral analyses, 13C and 1H one and two-dimensional NMR spectra illustrated in drawings filed herewith. The isomer is preferably isolated from cinnamon (Cinnamomum cassia). A preferred method of isolation of the present invention involves initial extraction of the novel tetrameric, type A proanthocyanidin isomer together with phenolic compounds in an ethyl alcohol extractant. The extract is subsequently processed using countercurrent chromatography and normal and HPLC techniques and the tetrameric isomer of the present invention is isolated thereby. Uses of the inventive and novel tetrametric isomer as an anti-inflammatory nutritional supplement, antioxidant, antimicrobial agent, treatment for polycystic ovarian syndrome, and glucose maintenance and insulin sensitizing agents are contemplated.

### BRIEF DESCRIPTION OF THE DRAWINGS

The accompanying drawings, which are incorporated herein and form a part of the specification, illustrate non-limiting embodiments of the present invention, and together with the description, serve to explain the principles of the invention. In the drawings:

FIG. 1 is a representation based on a traditional ball and stick model of the tetramer isomer of the presentation invention isolated from Cinnamomum cassia;

FIG. 2 is a two-dimensional representation of the isolated tetramer isomer shown in FIG. 1;

FIG. 3 is a rotamer of the isolated tetramer isomer of FIGS. 1 and 2, upon which long-range HMBC correlations have been superimposed;

FIG. 4 is another rotamer of the isolated tetramer isomer of FIGS. 1 and 2, upon which assignments have been superimposed;

FIG. 5 is a mass spectrometer analysis of the isolated tetramer isomer of the present invention which includes a 1153 m/Z peak;

FIG. 6 is a mass spectrometer analysis of the spectra of 1153 m/z peak of FIG. 5;

FIG. 7 illustrates a trace A which represents the 20-100 ppm 13C NMR spectra for the isolated tetramer isomer of the present invention obtained using Distortionless Enhancement by Polarization Transfer (DEPT); and

FIG. 8 illustrates Heteronuclear Single Quantum Coherence (HSQC) (1-bond marked with *) and Heteronuclear Multiple Quantum Coherence (HMQC) (2-, 3- and 4-bond) correlations for the isolated tetramer isomer of the present invention.

### DESCRIPTION OF THE INVENTION

The present invention relates to a novel, isolated tetrameric type A proanthocyanidin having a formula of C₆₀H₄₈O₂₄, according to the mass spectral analyses, 13C and 1H NMR spectra illustrated in drawings filed herewith. The isomer is most preferably isolated from cinnamon (Cinnamomum cassia.)

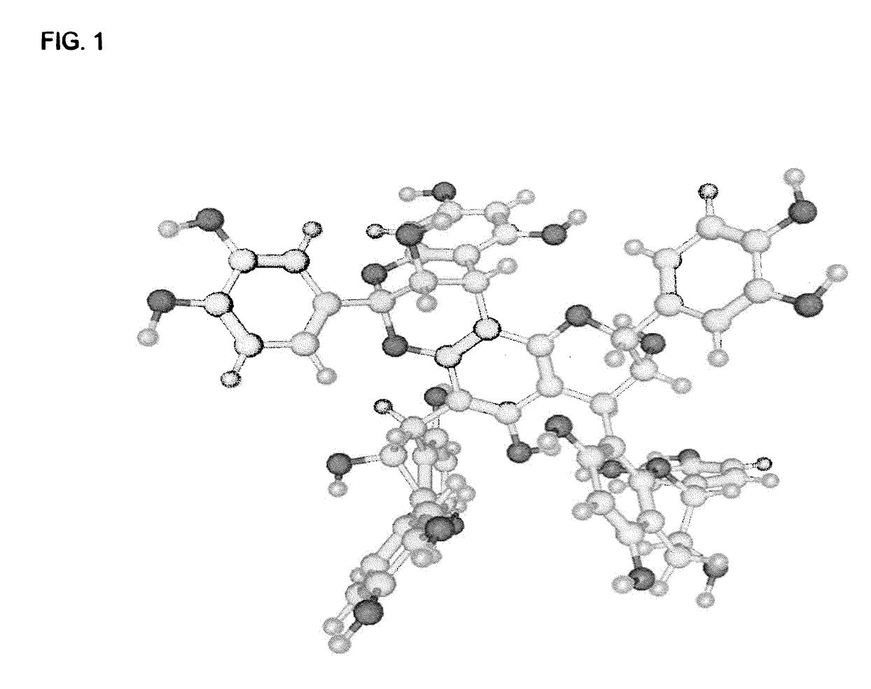

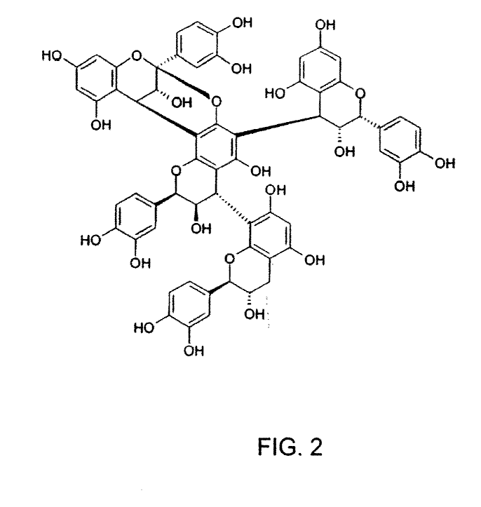

More particularly, FIG. 1 is a stick and ball model of the present invention and FIG. 2 is a 2-dimensional representation of the novel, isolated tetrameric type A proanthocyanidin of the present invention having a formula of C₆₀H₄₈O₂₄ which is reproduced below:

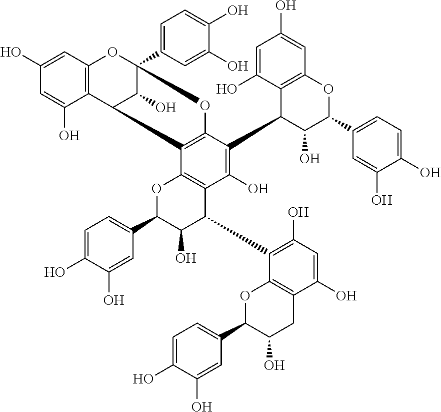

The stick and ball model shown in FIG. 1 was developed based on NMR spectra and MSMS analyses and also relates to pharmaceutical compositions containing the isolated tetrameric type A novel proanthocyanidin having a formula of C₆₀H₄₈O₂₄, according to the structures, HPLC analysis, C NMR spectra and H NMR spectra illustrated in drawings filed herewith.

The present invention also encompasses use of the above-identified compositions as a pharmaceutical and as a nutraceutical, for oral ingestion alone or with other components.

This invention provides methods for isolating the novel isomer of the present invention from phenolic-enriched compositions extracted from cinnamon. Most preferably, the isolated tetrameric type A novel proanthocyanidin having a formula of C₆₀H₄₈O₂₄ is extracted with other phenols in an ethyl alcohol extract. Extractions using acetone and/or other alcohols, including methanol and propanol in the presence of limited amounts of water, are also acceptable. Details of one preferred extraction methodology appropriate to and preceding the isolation techniques of the present invention are described in co-pending U.S. Publication No. 20060073220, which is incorporated herein in its entirety. The extract is then processed using counter-current chromatography and normal and reversed-phase high performance liquid chromatography. One exemplary counter-current chromatography technique is described in an article by N. Kohler, et al., Preparative Isolation Of Procyanidins From Grape Seed Extracts By High-Speed Counter-Current Chromatography, J. Chromatography A, 1177 (2008) 114-125, Nov. 17, 2007, incorporated herein by this reference. The isolated compound obtained via the aforementioned processes was then analyzed by a combination of spectroscopic techniques, including mass spectrometry and NMR.

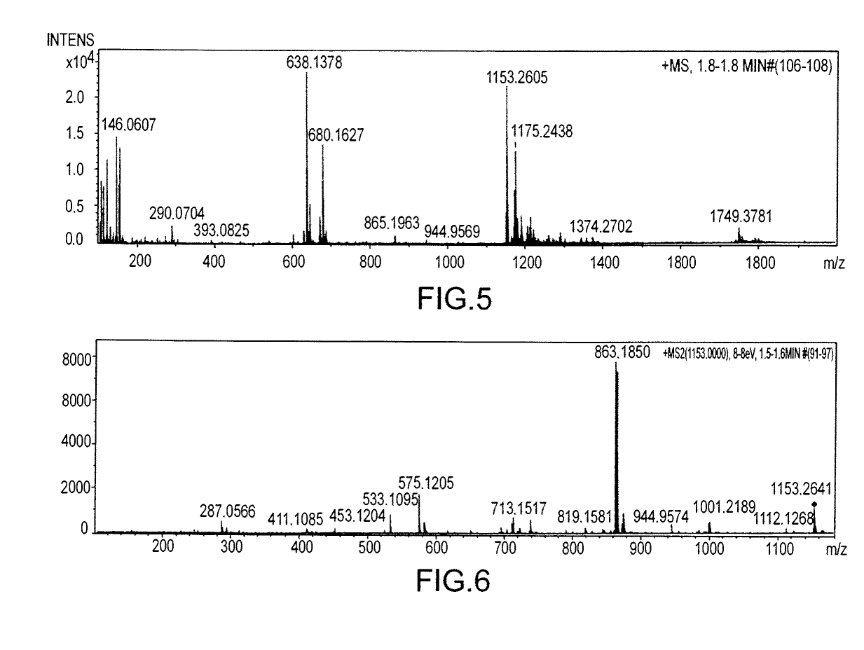

FIG. 5 shows high resolution mass spectrometer analysis of the isolated tetramer isomer of the present invention which includes a 1153 m/Z peak. The data generated by this analysis provides the exact mass of the isolated tetramer isomer of the present invention. FIG. 6 shows further details of a high resolution mass spectrometer analysis of the dominant fragment tied to the 1153 m/Z peak.

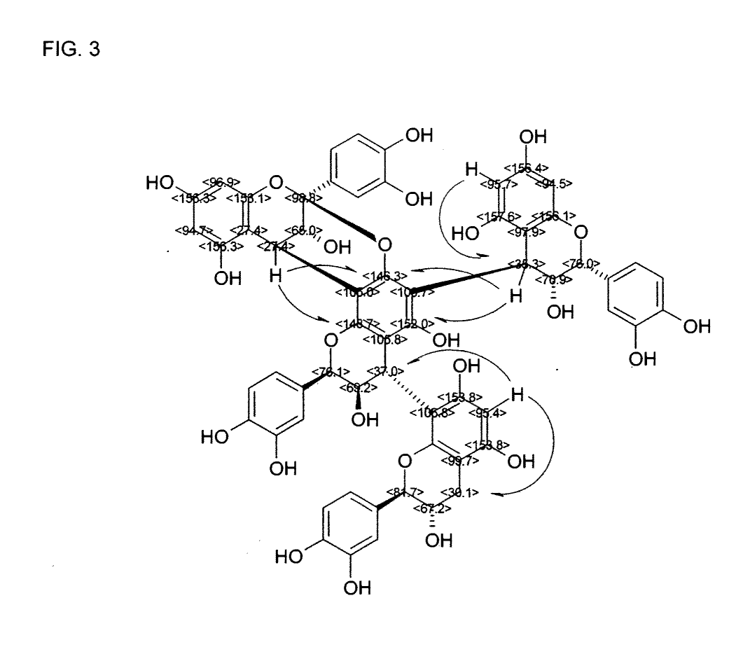

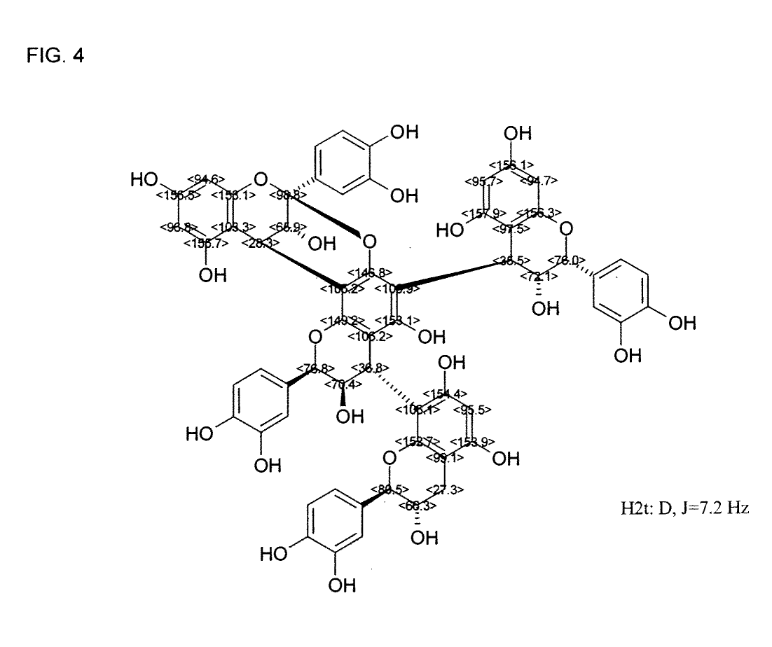

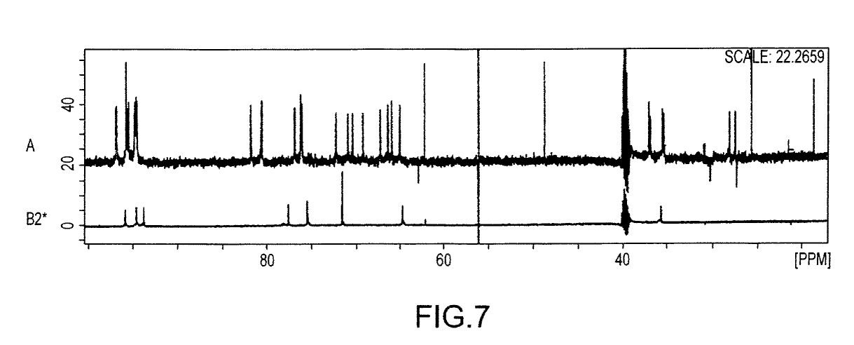

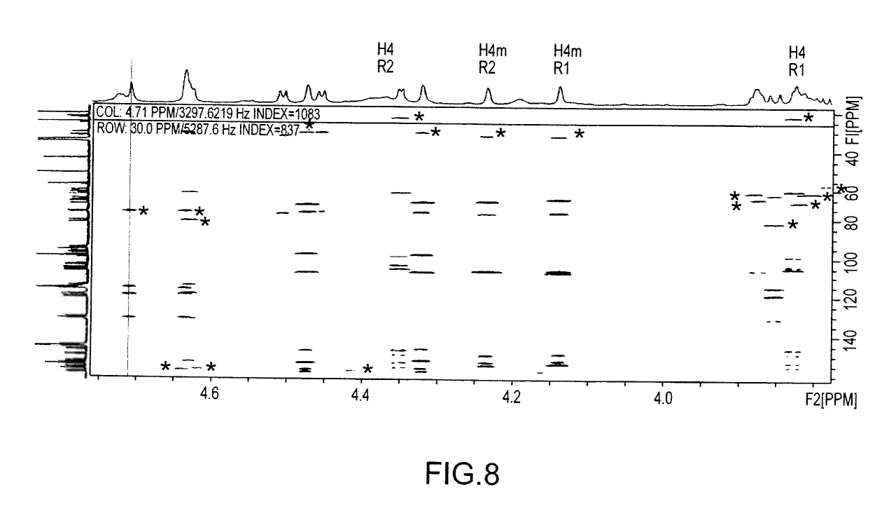

Trace A of FIG. 7 represents the 20-100 pm NMR 13C spectra for the isolated tetramer isomer of the present invention compared to a simpler reference procyanidin B2. The data obtained from this NMR analysis was used, in combination with the mass spectrometry data described above, to calculate the relationships of the atoms and moieties shown in FIGS. 3 and 4, from which the visually simpler representations shown in FIGS. 1 and 2 were based.

Note that the terms “phenols” and “phenolic compounds” are used interchangeably herein and include monomeric, oligomeric and polymeric compounds having one or more phenolic groups, and include, but are not limited to, anthocyanins, proanthocyanidins, and flavonoids. As used herein, the term “phenolic-enriched composition” refers to a composition enriched in one or more phenolic compounds and having substantially depleted levels of polar non-phenolic compounds present in crude extracts of plants, fruits, berries, and vegetables. Examples of such polar non-phenolic compounds include, but are not limited to, sugars, cellulose, pectin, amino acids, proteins, nucleic acids, and water.

As reported in U.S. Publication No. 20060073220, phenolic-enriched compositions possess a range of biological activities. For example, the compositions of this invention were found to have antiviral activities, with the compositions described therein to be used either alone or in combination with other antiviral agents to prevent and/or treat diseases induced by or complicated with viral infections from viruses including, but not limited to, influenza A, B, and C, parainfluenza virus, adenovirus type 1, Punta Toro Virus A, Herpes simplex virus I and II, rhinovirus, West Nile virus, Varicella-zoster virus and measles virus.

Compositions containing the isolated and novel tetramer isomer of the present invention are expected to have substantially greater antiviral activities than the compositions described in than U.S. Publication No. 20060073220. Daily dosages of 0.1 to 300 mg per day of the isomer described herein are contemplated, with a preferred range of 0.5 to 150 mg per day expected to be efficacious.

Phenolic-enriched compositions have also been investigated as anti-inflammatory substances due to their inhibition of cyclooxygenase activity. It has been shown that it is desirable for anti-inflammatory substances to be selective for COX-2 inhibition rather than COX-1 inhibition. Accordingly, another aspect of phenolic-enriched compositions relates to methods of treating inflammatory diseases in mammals comprising administering a therapeutically effective amount of a phenolic-enriched composition, polar proanthocyanidin-enriched composition, or a non-polar proanthocyanidin-enriched composition of this invention. For example, phenolic-enriched compositions described in U.S. Publication No. 20060073220 were found to have high COX-2/COX-1 inhibition selectivity and an IC₅₀ of 108 μg/mL.

Compositions containing the isolated and novel tetramer isomer of the present invention are expected to have substantially greater anti-inflammatory activity than the compositions described in than U.S. Publication No. 20060073220. Single daily dosages of 0.1 to 300 mg per day are contemplated.

The compound of the present invention can be used either alone or in combination with other anti-inflammatory agents to prevent or inhibit inflammatory responses. Such responses may be caused by conditions or diseases including, but not limited to, osteoarthritis, allergenic rhinitis, cardiovascular disease, upper respiratory diseases, wound infections, neuritis and hepatitis.

It is also known that proanthocyanidins isolated from cranberries and blueberries inhibit bacteria from attaching to the bladder wall, thereby reducing the potential for maladies such as urinary tract infections (Howell, A. B., et al., New England J Med., 339:1085-1086 (1998)). It has been postulated that proanthocyanidins exert their effect by inhibiting the adhesion of bacteria. U.S. Publication No. 20060073220 describes a method of preventing or treating urogenital infections in a mammal comprising administering an effective amount of a phenolic-enriched composition, polar proanthocyanidin-enriched composition, or a non-polar proantho-cyanidin-enriched composition of this invention in an amount sufficient to prevent, reduce or eliminate the symptoms associated with such infections.

Compositions containing the isolated and novel tetramer of the present invention are expected to have substantially greater anti-bacterial activity, and anti-microbial activity, more generally, than the compositions described in than U.S. Publication No. 20060073220. Daily dosages of 0.1 to 300 mg per day are contemplated.

It is further known that proanthocyanidins are potent antioxidants. For example, the antioxidant effects of proanthocyanidins are presumed to account for many of their benefits on the cardiovascular and immune systems. Accordingly, use of the isolated tetrameric, type A isomer of the present invention as an antioxidant is contemplated. Compositions containing the isolated and novel tetrameric isomer of the present invention may also be combined with antioxidative agents, including but not limited to, resveratrol and other pterostilbenes, tea extracts, vitamins A, C, D, E, beta-carotene, various anthocyanidins, and flavonoids, as well as selenium.

The tetrameric isomer of the present invention may be used as a dietary supplement (e.g., dietary antioxidants) and for the treatment of disorders in humans and mammals. Compositions containing the isolated and novel tetramer of the present invention are expected to have improved antioxidant capability. Single doses of 2.5 to 150 mg/day of the isomer described herein are expected to be efficacious. For this reason, compositions of this invention may be used for improving visual acuity and for treating circulatory disorders, diabetes, and ulcers. In particular use as an insulin sensitizing agent, as an agent for glucose maintenance, and for use in treating polycycstic ovarian syndrome (when administered in a therapeutically effective dose) are contemplated.

Compositions containing the isolated and novel tetramer of the present invention may also be combined with immunoactive agents, including but not limited to, arabinogalactan, curcumenoids, species of Echinacea, vitamins, minerals, polysaccharides and astragalus, and the isolated and novel tetramer of the present invention is expected to exhibit immunoactive activity. Compositions containing the isolated tetramer for the present invention can also be combined with antimutagenic agents including, but not limited to, green tea extracts, catechins, epicatechins, epigallocatechins, gallocatechins, and flavonoids. The isolated and novel tetramer of the present invention is expected to also exhibit antimutagenic activity.

Compositions containing the isolated and novel tetramer of the present invention may be formulated as pills, capsules, liquids, or tinctures. In formulating compositions according to this invention, a wide range of excipients may be used, the nature of which will depend, of course, on the intended mode of application of the composition. Examples of excipients include preservatives, carriers, and buffering, thickening, suspending, stabilizing, wetting, emulsifying, coloring and flavoring agents, and in particular carboxy vinyl polymers, propylene glycol, ethyl alcohol, water, cetyl alcohol, saturated vegetable triglycerides, fatty acid esters or propylene glycol, triethanolamine, glycerol, starch, sorbitol, carboxymethyl cellulose, lauryl sulphate, dicalcium phosphate, lecithin, etc.

The foregoing and other features, utilities and advantages of the invention will be apparent from the following more particular descriptions of preferred embodiments of the invention and as illustrated in the accompanying drawings and as particularly pointed out in the appended claims. More particularly, the foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. An isolated proanthocyanidin compound having the formula as shown, and pharmaceutically acceptable salts thereof:

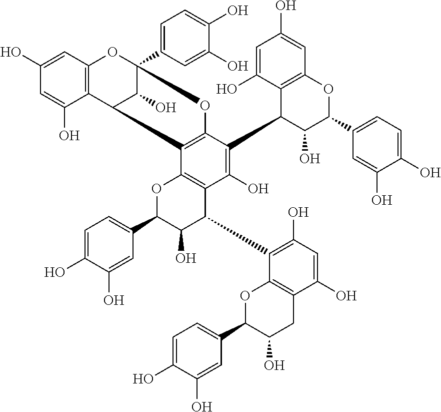

2. Use of the composition of claim 1 as an antimicrobial agent.

3. Use of the composition of claim 1 as an antioxidant.

4. Use of the composition of claim 1 as an anti-mutagenic agent.

5. Use of the composition of claim 1 as an insulin sensitizing agent.

6. Use of the composition of claim 1 for glucose maintenance.

7. Use of the composition of claim 1 for biofilm prevention.

8. A method for treating an polycystic ovarian syndrome comprising administration of a therapeutically effective dose of the compound of claim 1.

9. A method for treating cardiovascular disease comprising administration of a therapeutically effective dose of the compound of claim 1.

10. A pharmaceutical composition comprising a pharmaceutically acceptable carrier in admixture with the compound of claim 1.

11. Use of the composition of claim 10 as an insulin sensitizing agent.

12. Use of the composition of claim 10 for glucose maintenance.

13. Use of the composition of claim 10 as an antimicrobial agent.

14. A dietary supplement composition comprising a consumable carrier in admixture with the compound of claim 1.

15. Use of the composition of claim 14 as an antioxidant.

16. Use of the composition of claim 14 as an anti-mutagenic agent.

17. Use of the composition of claim 14 for biofilm prevention.

18. A method to obtain the compound of claim 1 from cinnamon, the method comprising processing cinnamon bark to obtain an extract comprising phenols including the compound of claim 1 and isolating the compound of claim 1 from the preparation.
